(19) United States
(12) Patent Application Publication
(10) Pub. No.: US 2025/0228532 A1
(43) Pub. Date: Jul. 17, 2025

(54) SAMPLE COLLECTION DEVICE

(71) Applicant: Quanta Vision Co., Ltd., New Taipei City (TW)

(72) Inventors: KUAN-CHIAO LIAO, New Taipei City (TW); YA-CHIEH YU, New Taipei City (TW); CHIH-YI HSU, New Taipei City (TW)

(21) Appl. No.: 18/414,943

(22) Filed: Jan. 17, 2024

(51) Int. Cl. A61B 10/00

(52) U.S. Cl. CPC A61B 10/0051; A61B 10/0096

(57) ABSTRACT

A sample collection device is provided, including: a container, having a chamber, including an opening and an outlet which are disposed on two opposite sides thereof, the opening, the outlet and the chamber communicated with one another, an inner wall of the container having a plurality of protrusion portions, the plurality of protrusion portions radially protruding into the chamber; a filtration unit, disposed within the container and between the chamber and the outlet; and a sampling unit, including a main body and a sample collection member made of deformed absorbent material, the sample collection member disposed on the main body, and the sample collection member being insertable into the chamber and radially squeezed by the plurality of protrusion portions.

### BACKGROUND OF THE INVENTION

### Field of the Invention

The present invention relates to a sample collection device which is designed for collecting specimens.

### Description of the Prior Art

Many infectious rapid test devices generally collect specimens from oral cavity or nasal cavity for testing nowadays. The current specimen collection methods do not offer an accurate and effective way to quantify the specimen. Therefore, if quantitative analysis is required, additional quantitative experiments must be performed after specimen collection; however, this process is very complicated and inconvenient.

Furthermore, the proportion between the concentration of the specimen and the buffer solution in the rapid test reagent affects the result. Therefore, if accurate quantification cannot be achieved, it will inevitably lead to erroneous results.

The conventional rapid test devices include a sample-dropping rapid test device, a rapid test device with a volcano-shaped opening, a one-piece molded test device, and a squeezing machine. The sample-dropping rapid test device uses a dropper to drop the testing solution into the cassette; thus, it is difficult to quantify the testing solution due to the varying pressure applied while pressing the dropper. The rapid test device with a volcano-shaped opening is hard for user to control the squeezing pressure, resulting in poor quantification. It is impossible to quantity the testing solution by the one-piece molded test device, because a saliva sponge and a detection cassette of the one-piece molded test device are integrally formed. The squeezing machine uses a cassette to collect the saliva. The cassette containing saliva is then placed into the squeezing machine for compression. However, the squeezing machine has disadvantages in high equipment cost and large size; thus, it is unsuitable for portable.

The present invention is, therefore, arisen to obviate or at least mitigate the above-mentioned disadvantages.

### SUMMARY OF THE INVENTION

The main object of the present invention is to provide a sample collection device, which provides sampling and quantification of specimens.

To achieve the above and other objects, a sample collection device is provided, including: a container, having a chamber, including an opening and an outlet which are disposed on two opposite sides thereof, the opening, the outlet and the chamber communicated with one another, an inner wall of the container having a plurality of protrusion portions, the plurality of protrusion portions radially protruding into the chamber; a filtration unit, disposed within the container and between the chamber and the outlet; and a sampling unit, including a main body and a sample collection member made of deformed absorbent material, and the sample collection member disposed on the main body, the sample collection member being insertable into the chamber and radially squeezed by the plurality of protrusion portions.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a stereogram of a first exemplary embodiment of the present invention;

FIG. 2 is a breakdown drawing of a first exemplary embodiment of the present invention;

FIG. 3 is a top plan view of a first exemplary embodiment of the present invention;

FIG. 4 is a stereogram of a tube body of a first exemplary embodiment of the present invention;

FIGS. 5-7 are drawings of a first exemplary embodiment of the present invention in use;

FIG. 8 is a stereogram of a tube body of a second exemplary embodiment of the present invention; and

FIG. 9 is a cross-sectional view of a second exemplary embodiment of the present invention.

### DETAILED DESCRIPTION OF THE PREFERRED EMBODIMENTS

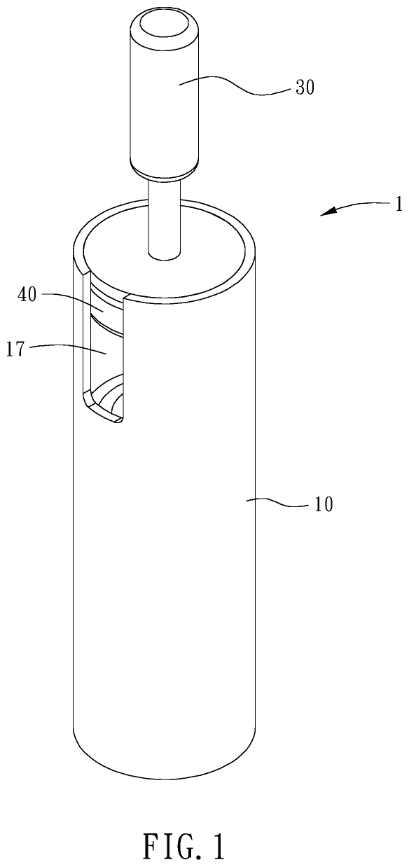

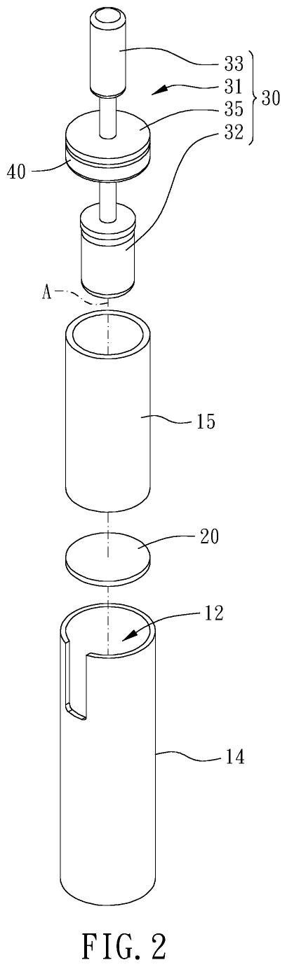

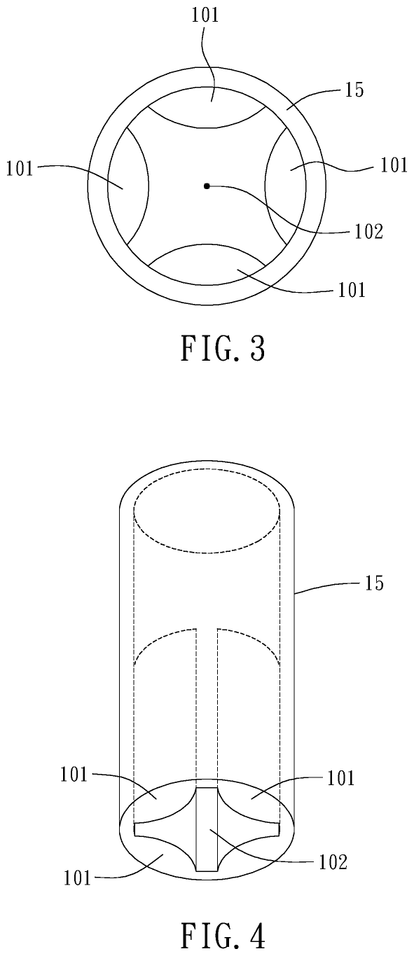

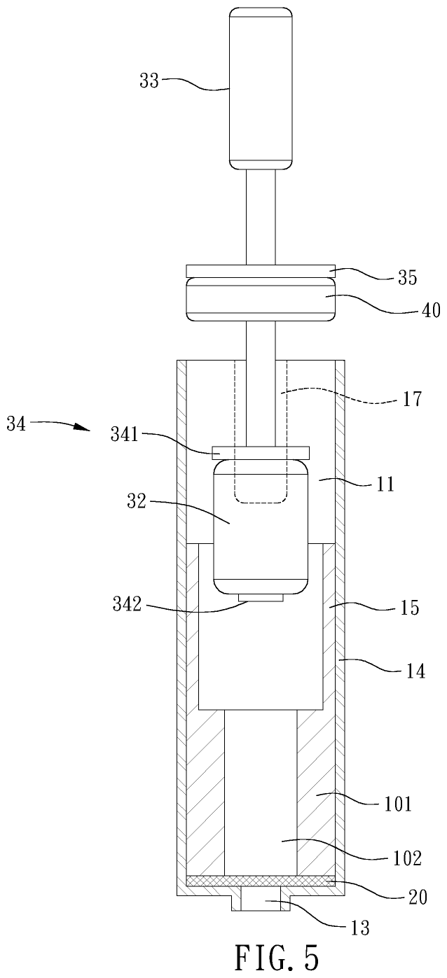

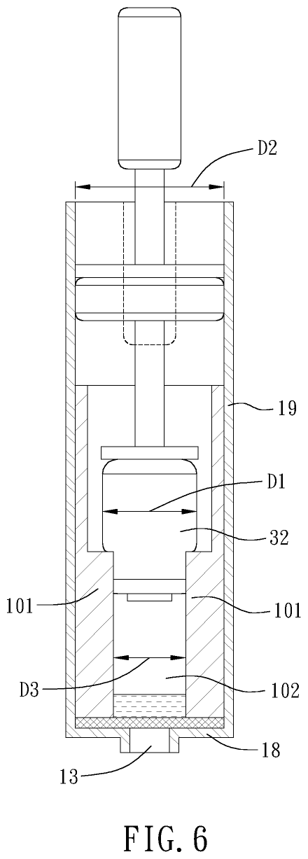

Please refer to FIGS. 1 to 7 for a first exemplary embodiment of the present invention. A sample collection device 1 of the present invention includes a container 10, a filtration unit 20 and a sampling unit 30.

The container 10 has a chamber 11 and includes an opening 12 and an outlet 13 which are disposed on two opposite sides thereof. The opening 12, the outlet 13 and the chamber 11 are communicated with one another. An inner wall of the container 10 has a plurality of protrusion portions 101. The plurality of protrusion portions 101 are radially protruding into the chamber 11. The filtration unit 20 is disposed within the container 10 and between the chamber 11 and the outlet 13. The filtration unit 20 is a filter paper. The sampling unit 30 includes a main body 31 and a sample collection member 32 made of deformed absorbent material. The sample collection member 32 is disposed on the main body 31, and the sample collection member 32 is insertable into the chamber 11 and radially squeezed by the plurality of protrusion portions 101 to collect a specimen. The sample collection member 32 can be a sponge, a swab, or other materials capable of absorbing liquid. The sample collection member 30 absorbs a sample, such as saliva sample and other fluid sample.

Therefore, the specimen containing on the sample collection member 32 can be squeezed out by the plurality of protrusion portions 101, and the plurality of protrusion portions 101 can apply a fixed depth and range of compression onto the sample collection member 32 to achieve the collection of specimen and quantification. Moreover, the impurities (such as food residue or other contaminants) from the specimen can be filtered by the filtration unit 20.

The specimen can flow out of the chamber 11 through the outlet 13.

The sample collection device 1 includes at least three said protrusion portions 101. The chamber 11 defines a central axis A, and the at least three said protrusion portions 101 disposed circumferentially on the inner wall of the container 10 and arranged around the central axis A. In this embodiment, the sample collection device 1 includes at least four said protrusion portions 101. Besides, the plurality of protrusion portions 101 are circumferentially spaced; thus, the sample collection member 32 can be squeezed evenly.

Each of the plurality of protrusion portions 101 is an arcuate convex protruding radially. Specifically, a top end of each of the plurality of protrusion portions 101 has a convex arc surface. Therefore, the sample collection member 32 can be smoothly inserted into a space which forms between the plurality of protrusion portions 101 and can be squeezed by the plurality of protrusion portions 101, and to ensure that the sample collection member 32 remains undamaged when axially moving and rotating relative to the multiple protrusion portions 101.

The plurality of protrusion portions 101 form a passage hole 102 therebetween, the passage hole 102 is communicated with the outlet 13 and the chamber 11, the sample collection member 32 is movable through the passage hole 102, an outer diameter D1 of the sample collection member 32 is less than an opening size D2 of the opening 12 and greater than a minimum aperture size D3 of the passage hole 102; thus, the sample collection member 32 can be smoothly passed through the opening 12 without being squeezed until it is inserted into the passage hole 102, so as to avoid splashing the specimen from the opening 12 and the specimen can flow out through the outlet 13.

The container 10 includes a cylinder body 14 and a tube body 15. The cylinder body 14 is sleeved on the tube body 15, and the tube body 15 and the plurality of protrusion portions 101 are integrally formed. The cylinder body 14 and the tube body 15 can be connected with each other (such as by welding, adhesive bonding or fastening) to facilitate manufacturing processes.

The tube body 15 presses (down) the filtration unit 20, so as to secure the filtration unit 20 firmly.

The container 10 includes a bottom wall 18 and a peripheral wall 19. One end of the peripheral wall 19 is circumferentially connected with the bottom wall 18, another end of the peripheral wall 19 axially extends away from the bottom wall 18 and forms the opening 12, and the peripheral wall 19 encloses the chamber 11. The container 10 defines a first height H1, the first height H1 is a distance between the opening 12 and the bottom wall 18. Each of the at least four said protrusion portions 101 defines a second height H2 extending axially, the second height H2 is smaller than the first height H1. In this embodiment, the second height H2 is smaller than 0.6 times the first height H1. Preferably, the container 10 defines a center line extending radially through a center of the container, and each of the plurality of protrusion portions 101 is lower than the center line. Therefore, the sampling unit 30 needs to be inserted into the chamber 11 to a certain depth to be squeezed by the plurality of protrusion portions 101, so as to prevent the specimen on the sample collection member 32 from splashing out through the opening 12 and adhering to the user's hands after the sample collection member 32 is squeezed.

The sampling unit 30 further includes a sealing member 40. The sealing member 40 is assembled to the main body 31. The sealing member 40 seals around an inner wall of the chamber 11 to block the chamber 11, and to achieve an airtight seal within the chamber 11. The sealing member 40 is an O-ring. When the sealing member 40 is moved downward within the chamber 11, the air within the chamber 11 will be compressed to prevent the specimen from flowing out backwards and adhering to the user's hands.

Two opposite end portions of the main body 31 includes a grip portion 33 and a connection portion 34, the main body 31 radially protrudes a cover portion 35 which is disposed between the grip portion 33 and the connection portion 34, and the sealing member 40 is assembled to the main body 31 and positioned beneath the cover portion 35. The cover portion 35 covers the opening 12 to provide multiple layers of protection. The specimen containing on the sample collection member 32 can be blocked by the cover portion 35 and the sealing member 40 to prevent from splashing out of the opening 12 when the sample collection member 32 is squeezed by the protrusion portions 101. The grip portion 33 is designed for handholding to facilitate the stable insertion of the sample collection member 32 through the opening 12 into the chamber 11. Besides, an outer diameter of the cover portion 35 is equal to or less than the opening size D2 of the opening 12, and the cover portion 35 is passed through the opening 12 to be placed within the chamber 11, the sampling unit 30 can be guided for stable axial movement within the chamber 11 without swaying by the cover portion 35, so that the plurality of protrusion portions 101 can apply a fixed depth and range of compression onto the sample collection member 32.

The peripheral wall 19 of the container 10 has a through hole 17 which is radially disposed therethrough and adjacent to the opening 12, the through hole 17 is communicated with the chamber 11, and the sealing member 40 axially passes through the through hole 17; therefore, the chamber 11 will reach an airtight effect only when the sealing member 40, which is disposed within the chamber 11, axially passes through the through hole 17 to be located beneath the through hole 17, so that the sealing member 40 can be tightly sealed with the container 10, and smoothly passed through the opening 12 into the chamber 11.

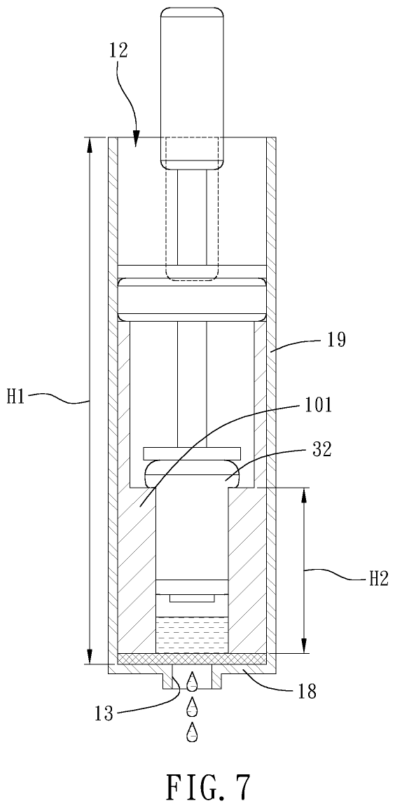

The sealing member 40 is axially blocked by a top end of the tube body 15, as shown in FIG. 7, so as to restrict a downward movement distance of the sealing member 40 within the chamber 11, allowing the sealing member 40 to compress a precisely quantified volume of the specimen through the outlet 13 during the pushing process.

The connection portion 34 is I-shaped, the connection portion 34 includes a first flange 341 and a second flange 342 which are disposed separately, and the sample collection member 32 is disposed between the first flange 341 and the second flange 342, so as to restrict the sample collection member 32 between the first flange 341 and the second flange 342.

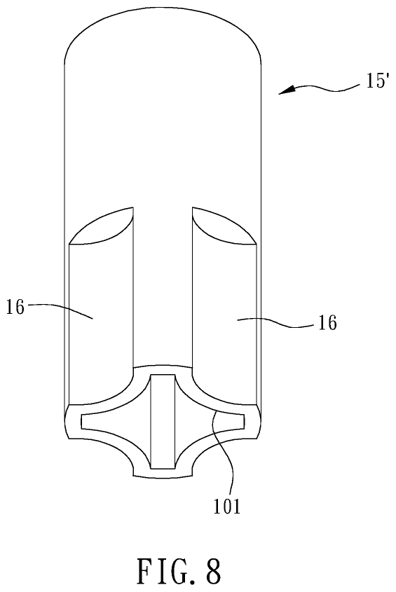

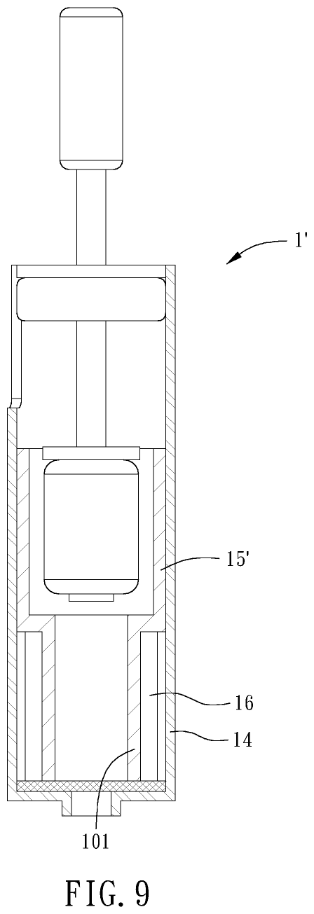

Please refer to FIGS. 8 to 9 for a second exemplary embodiment of the present invention. The tube body 15′ of the sample collection device 1′ is deformed inwardly to integrally protrude the plurality of protrusion portions 101, and an external portion of the tube body 15′ forms a plurality of concave portions 16 which correspond to the plurality of protrusion portions 10. Therefore, a plurality of spaces, which correspond to the plurality of concave portions 16, are formed between the tube body 15′ and the cylinder body 14, so as to reduce a contact area between the tube body 15′ and the cylinder body 14 to facilitate the easy assembly of the tube body 15′ and the cylinder body 14 and to offer advantages in material saving and lightweight construction.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A sample collection device, including:
a container, having a chamber, including an opening and an outlet which are disposed on two opposite sides thereof, the opening, the outlet and the chamber communicated with one another, an inner wall of the container having a plurality of protrusion portions, the plurality of protrusion portions radially protruding into the chamber;
a filtration unit, disposed within the container and between the chamber and the outlet; and
a sampling unit, including a main body and a sample collection member made of deformed absorbent material, the sample collection member disposed on the main body, and the sample collection member being insertable into the chamber and radially squeezed by the plurality of protrusion portions.

2. The sample collection device of claim 1, including at least three said protrusion portions, the chamber defining a central axis, and the at least three said protrusion portions disposed circumferentially on the inner wall of the container and arranged around the central axis.

3. The sample collection device of claim 2, wherein the plurality of protrusion portions are circumferentially spaced.

4. The sample collection device of claim 1, wherein each of the plurality of protrusion portions is an arcuate convex protruding radially.

5. The sample collection device of claim 1, wherein the container includes a cylinder body and a tube body, the cylinder body is sleeved on the tube body, and the tube body and the plurality of protrusion portions are integrally formed.

6. The sample collection device of claim 5, wherein the tube body presses the filtration unit.

7. The sample collection device of claim 5, wherein the tube body is deformed inwardly to integrally protrude the plurality of protrusion portions, and an external portion of the tube body forms a plurality of concave portions which correspond to the plurality of protrusion portions.

8. The sample collection device of claim 5, wherein the sampling unit further includes a sealing member, the sealing member is assembled to the main body, the sealing member seals around an inner wall of the chamber to block the chamber, and the sealing member is axially blocked by a top end of the tube body.

9. The sample collection device of claim 8, wherein a peripheral wall of the container has a through hole which is radially disposed therethrough and adjacent to the opening, the through hole is communicated with the chamber, and the sealing member axially passes through the through hole.

10. The sample collection device of claim 9, including at least four said protrusion portions, the chamber defining a central axis, and the at least four said protrusion portions disposed circumferentially on the inner wall of the container and arranged around the central axis; wherein the plurality of protrusion portions are circumferentially spaced; each of the plurality of protrusion portions is an arcuate convex protruding radially; the container includes a bottom wall, one end of the peripheral wall is circumferentially connected with the bottom wall, another end of the peripheral wall axially extends away from the bottom wall and forms the opening, the peripheral wall encloses the chamber, the container defines a first height, the first height is a distance between the opening and the bottom wall, each of the at least four said protrusion portions defines a second height extending axially, the second height is less than the first height; the second height is smaller than 0.6 times the first height; the tube body presses the filtration unit; the plurality of protrusion portions form a passage hole therebetween, the passage hole is communicated with the outlet and the chamber, the sample collection member is movable through the passage hole, an outer diameter of the sample collection member is less than an opening size of the opening and greater than a minimum aperture size of the passage hole; two opposite end portions of the main body includes a grip portion and a connection portion, the main body radially protrudes a cover portion which is disposed between the grip portion and the connection portion, and the sealing member is assembled to the main body and positioned beneath the cover portion, the cover portion covers the opening; an outer diameter of the cover portion is equal to or less than the opening size of the opening, and the cover portion is passed through the opening to be placed within the chamber; the connection portion is I-shaped, the connection portion includes a first flange and a second flange which are disposed separately, the sample collection member is disposed between the first flange and the second flange; and the container defines a center line extending radially through a center of the container, and each of the plurality of protrusion portions is lower than the center line.
